(19) United States
(12) Patent Application Publication
(10) Pub. No.: US 2011/0275885 A1
(43) Pub. Date: Nov. 10, 2011

(54) RECOVERABLE INTRA-UTERINE SYSTEM

(75) Inventor: Nicolas Bouche, Les Paccots (CH)

(73) Assignee: Anecova SA, Lausanne (CH)

(21) Appl. No.: 12/735,811

(22) PCT Filed: Feb. 13, 2009
(86) PCT No.: PCT/IB2009/000482

§ 371 Date: May 17, 2011

(30) Foreign Application Priority Data
Feb. 20, 2008 (FR) 0851086

(51) Int. Cl. A61B 17/435; A61B 17/43

(52) U.S. Cl. 600/34; 600/35

(57) ABSTRACT

A recoverable intra-uterine system comprises a receptacle (10) designed to contain one or more elements chosen from the group comprising an embryo, male and/or female gametes, a fertilized oocyte, an unfertilized ovum, and a combination of these elements, and a retaining device (20) for retaining the recoverable intra-uterine system in the uterus. The retaining device (20) comprises a deformable element (21) designed to be retained by bearing in the neck of the uterus. Use in artificial insemination techniques.

The present invention concerns a recoverable intra-uterine system.

Generally speaking, it concerns the stabilization and the retention in position of a recoverable intra-uterine system used in artificial insemination.

Intra-uterine systems implantable and recoverable naturally have been known for a number of years.

Generally speaking, these systems may be classified into three large families according to their indication or their mode of action. Of these, devices having a contraceptive indication, such as the coil, represent the great majority of cases and constitute the first category.

The second category consists of capsules loaded with a therapeutic substance and placed in the uterine cavity for diffusion and action in the organism.

Finally, the third category consists of intra-uterine devices indicated for in vivo assistance in artificial insemination. Thus there is known a intra-uterine device as described in particular in the document WO 03/011200 intended to be placed in the uterine cavity for a period between a few hours and a few days, enabling pre-implantation development of an embryo in vivo.

For each of these categories of devices, it is of primary importance that the object placed in the uterine cavity naturally is able to remain positioned at the particular location for the indicated duration and then recovered naturally.

To meet these specifications, the devices concerned must respond to two a priori contradictory constraints. On the one hand, these devices must be thin and compact in order to be placed naturally in the uterine cavity and then extracted naturally, which requires passing through the isthmus of the neck of the uterus. On the other hand, they must be sufficiently bulky to be stable in the uterine cavity without being expelled by natural contractions of the uterus.

Numerous variants and shapes have been proposed in the prior art to respond to these two constraints but oriented exclusively to applications in the first and second categories of devices referred to above.

Now, for the third category of devices used for in vivo artificial insemination, an additional constraint appears as it is of primary importance to preserve the integrity of the endometrial wall. If implantation of the embryo takes place during the same menstrual cycle of the patient, the endometrium must not be degraded before implantation of the embryo.

In particular, existing stabilization systems in the field of contraception, and notably for retaining a coil in position in the uterus, are not satisfactory. They may damage the endometrium, not only when the device is retained in position in the uterine cavity, by exerting a constant pressure against the endometrium (causing contraction phenomena, possibly with hormonal activation), but also during withdrawal of the device, the retaining elements rubbing on the endometrial wall over a great distance, thus causing microlesions, inflammation or bleeding.

Moreover, the document WO 03/011200 describes an intra-uterine device carrying stabilizing fins at a distal end of the receptacle. These stabilizing fins are liable to damage the endometrial wall, especially during withdrawal of the device, and thereby to reduce the chances of successful subsequent implantation of the embryo.

An object of the present invention is to propose a recoverable intra-uterine system that makes it possible to solve the aforementioned problems and especially to guarantee that the system is retained in position in the uterine cavity at the same time as preserving the integrity of the endometrium.

To this end, the present invention concerns a recoverable intra-uterine system comprising a receptacle adapted to contain one or more elements chosen from the group comprising an embryo, male and/or female gametes, a fertilized oocyte, a non-fertilized ovum and a combination of these elements, and a device for retaining the recoverable intra-uterine system in the uterus.

According to the invention, the retaining device comprises a deformable element adapted to assume at least two positions:
an introduction position in which said deformable element is substantially rectilinear and adapted to be accommodated in a catheter; and
a retaining position in which said deformable element is adapted to cooperate in bearing engagement with a wall of the neck of the uterus.

Thus the receptacle introduced into the uterus is retained in position in the neck of the uterus in order to limit contact with the endometrial wall of the body of the uterus.

Accordingly, as much when introducing the recoverable intra-uterine receptacle as when withdrawing it, there is only very limited, or even non-existent, rubbing or risk of damaging the wall of the endometrium beyond the isthmus.

Thanks to the deformation of the retaining device, the whole of the system may be introduced into a traditional transfer catheter used to introduce the recoverable intra-uterine system into the uterine cavity beyond the neck of the uterus.

Thus the system may be introduced without risk of damaging the neck of the uterus or the endometrial wall by rubbing on them by using a transfer catheter of small inside and outside diameter adapted to pass through the neck of the uterus.

According to one particular feature of the invention, the deformable element is a spiral deformable elongate element.

Thus the contact between the spiral elongate element and the wall of the neck of the uterus is limited and the retaining device is not likely to obstruct the neck of the uterus and to impede existing flow of fluid between the uterus and the vagina.

According to one practical feature of the invention, the element of the retaining device is deformable between an introduction position and a retaining position by an elastic return force.

Thanks to the elastic return force, the retaining device is automatically placed in a retaining position in the neck of the uterus when the recoverable intra-uterine system is introduced into the uterine cavity and after withdrawal of the transfer catheter used for its introduction.

Thus the recoverable intra-uterine system is retained in position reliably and naturally without requiring additional manipulation by the practitioner.

Another aspect of the invention is a combination comprising a transfer catheter and an intra-uterine system in which the system is accommodated inside the catheter.

Thus the receptacle associated with the retaining device may be placed in the uterus thanks to the transfer catheter without risk of damaging the tissues.

Other features and advantages of the invention will become more apparent in the following description.

In the appended drawings, provided by way of nonlimiting example:

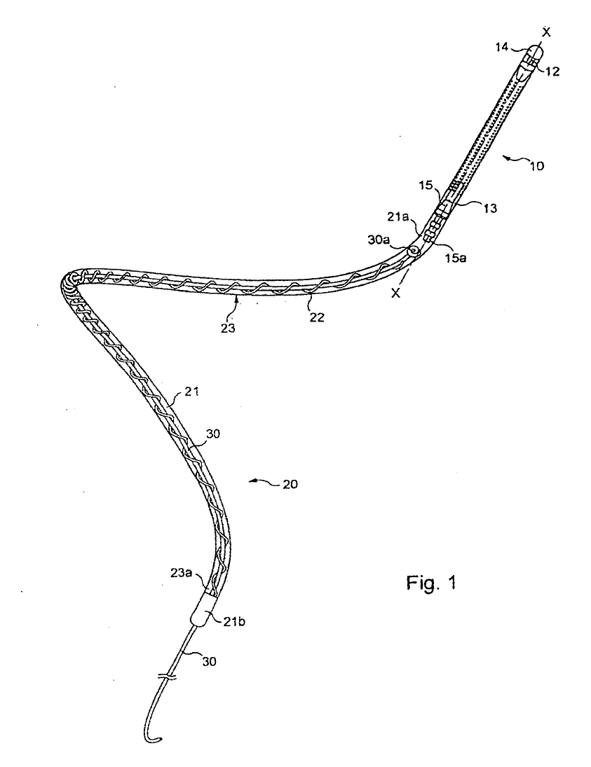

FIG. 1 is a diagrammatic view of a recoverable intra-uterine system of one embodiment of the invention;

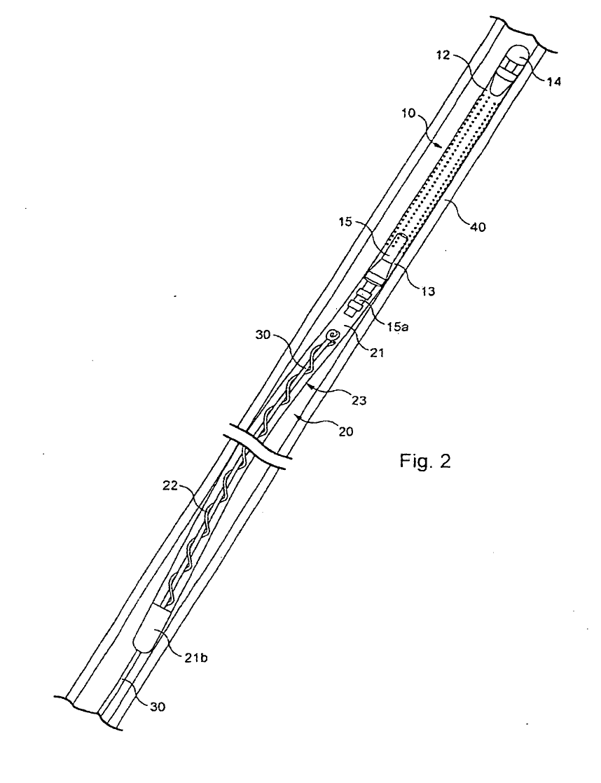

FIG. 2 is a diagrammatic view illustrating the mounting of a recoverable intra-uterine system from FIG. 1 in a transfer catheter;

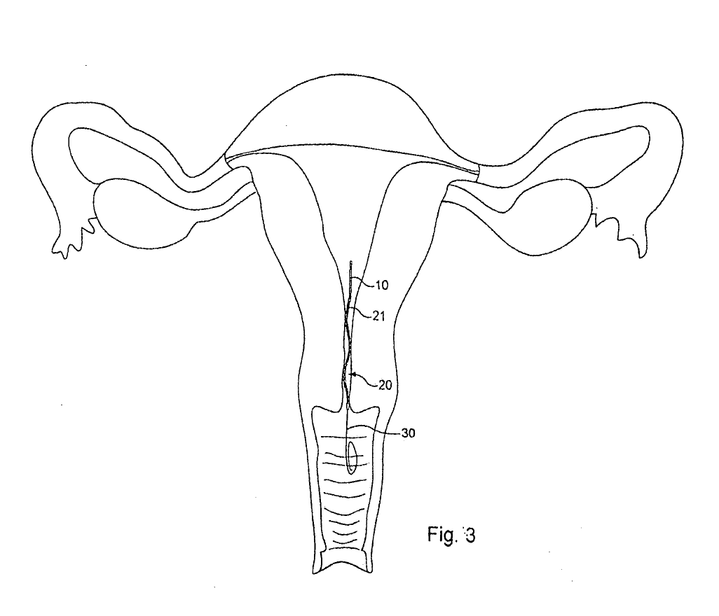

FIG. 3 is a diagrammatic view in section illustrating the positioning of the recoverable intra-uterine system in the uterine cavity.

As shown in FIG. 1, the recoverable intra-uterine system firstly comprises a receptacle 10 intended to be placed in the uterus.

This receptacle 10 is intended to be placed in the uterine cavity for a period between a few hours and a few days with a view to pre-implantation development of an embryo in vivo.

To this end, the receptacle 10 is adapted to contain an embryo, male and/or female gametes, a fertilized oocyte, a non-fertilized ovum or a combination of these various elements.

This receptacle 10 may be produced in silicone and provided with a series of perforations in its wall enabling interaction between the interior of the receptacle and the exterior medium in which the receptacle is placed, i.e. the uterine medium.

As clearly illustrated in FIG. 1, this receptacle 10 is of elongate shape along an axis X.

In this embodiment, the receptacle 10 consists of an elongate cylindrical tube in transparent silicone.

By way of indication, the length of the receptacle in the direction X may be substantially equal to 10 mm, the inside diameter of the receptacle 10 being between 0.4 and 0.5 mm inclusive and the outside diameter of the cylindrical receptacle 10 being between 0.7 and 0.8 mm inclusive.

This elongate receptacle 10 therefore has on its axis of introduction into a uterine cavity along the axis X a distal end 12 and a proximal end 13.

The receptacle 10 has at its distal end 12 a stopper 14, for example in titanium, fitted and fixed to the end of the receptacle 10 to block the latter.

The stopper 14 may be removed after use of the intra-uterine system in order to extract embryos and/or other elements that have been placed temporarily in the uterine cavity.

The elements withdrawn in this way, especially the embryo, can then be selected and re-implanted in the uterine cavity.

In a similar manner, the receptacle 10 has a proximal stopper 15 at its proximal end 13.

The receptacle 10 is also associated with a device 20 for retaining the intra-uterine system in the uterus. This retaining device 20 is at the proximal end 13 of the receptacle 10.

Generally speaking, this retaining device 20 comprises a deformable element 21 adapted to be held in the neck of the uterus by bearing engagement therewith.

In this embodiment, the deformable element 21 is a deformable spiral elongate member.

As clearly illustrated in FIG. 2, the retaining device 20 is adapted to assume an introduction position in which the deformable element 21 can be accommodated in a catheter 40.

In this position, the deformable element 21 is disposed in line with the receptacle 10, i.e. substantially aligned with it in the longitudinal direction X of the receptacle 10.

Moreover, the retaining device 20 is adapted to assume a retaining position as shown in FIG. 3 in which the deformable element 21 is adapted to cooperate with a wall of the neck of the uterus.

In this position, the deformable element is of substantially spiral shape.

In this embodiment, the deformable element 21 comprises a steel element 22 forming a rod covered with a protective sleeve 23.

The steel rod may be produced from a steel coil spring and extends inside the protective sleeve 23.

This protective sleeve 23 may be produced in biocompatible silicone tube.

The sleeve 23 housing the spring 22 has a length between 45 and 60 mm inclusive in the stretched configuration, i.e. when it is substantially aligned in the longitudinal direction X.

When it is at rest, i.e. spiral in shape, this sleeve 23 extends over a length between substantially 15 and 25 mm inclusive.

Here the deformable element 21 of the retaining device 20 is deformable between the introduction position illustrated in FIG. 2 and a free rest position in which the deformable element assumes the shape of a spiral because of an elastic return force.

Thus in the introduction position the deformable element 21 is constrained, against the elastic return force, to occupy a substantially rectilinear elongate configuration and in the rest position the deformable element 21 resumes an elongate spiral configuration because of the action of the elastic return force.

During its introduction into the uterus, the deformable element is limited to the rest position by the dimensions of the neck of the uterus so that the elastic return force constrains the deformable element 21 to come into contact with the walls of the neck of the uterus.

In practice, the spring 22 is introduced into the protective sleeve 23 and the combination is fastened to the proximal stopper 15 at the proximal end 13 of the receptacle 10.

The silicone protective sleeve 23 is preferably force-fitted onto a rod 15a of the proximal stopper 15 of the receptacle 10.

To obtain a spiral deformation of the deformable element 21, the combination consisting of the steel spring 22 and the protective sleeve 23 is wound in a spiral around a rod in order to mechanically deform the deformable element.

Furthermore, the recoverable intra-uterine system comprises a withdrawal thread 30 making it possible to withdraw the device from the uterus by traction. This withdrawal thread 30 may be in nylon and have a total length of 150 mm and a diameter of 0.1 mm.

As clearly illustrated in FIG. 1, this withdrawal thread extends inside the protective sleeve 23 and passes to the outside from the end 23a of the protective sleeve.

The withdrawal thread 30 is preferably inserted into the protective sleeve 23 at the same time as the steel element forming the spring 22.

The distal end part 21a of the elongate element 21 is preferably molded onto the proximal stopper 15 to strengthen the mounting of the protective sleeve 23 on the rod 15a of the proximal stopper 15.

By applying heat, the end 30a of the withdrawal thread 30 melts slightly inside the silicone protective sheath 23 to fix this withdrawal thread 30 to the inside of the protective sleeve 23 and to hold the spring element 22 in position.

Similarly, a molding operation is performed at the proximal end 21b of the elongate element 21 to fasten the withdrawal thread 30 to the silicone protective sleeve 23 over a portion of its length at this proximal end 21b.

This welds the nylon thread to the interior of the silicone protective sleeve 23 and thus fastens the protective sleeve 23 around the spring element 22 and the withdrawal thread 30.

The various positions that the retaining device of the intra-uterine system may assume during use are described next with reference to FIGS. 2 and 3.

In practice, the receptacle 10 is assembled to the retaining device 20 after introduction of the above elements, for example male and female gametes, into the receptacle.

During the introduction of the above elements, the distal stopper 14 is in place at the distal end 12 of the receptacle 10.

After introduction of the gametes, the stopper 15 is mounted at the end 13 of the receptacle 10 at the same time as mounting the retaining device 20 by fixing the protective sleeve 23 to this proximal stopper 15.

To enable the introduction of the intra-uterine system into the uterine cavity, a catheter 40 (shown in part in FIG. 2) used in the traditional way has the overall shape of an elongate cylindrical tube of appropriate length able to accommodate the whole of the intra-uterine system, i.e. in succession the receptacle 10 and the retaining device 20, as well as part of the withdrawal thread 30.

Thus the withdrawal thread 30 extends out of the transfer catheter 40.

By way of indication, a standard catheter has an inside diameter between 0.9 and 1.1 mm inclusive adapted to pass through the neck of the uterus.

In the introduction position shown in FIG. 2, the deformable element 21 is substantially rectilinear and adapted to be accommodated in the catheter.

The force producing elastic deformation of this deformable element 21 must not be too high to enable introduction of the retaining device 20 into the catheter by deformation and thereafter withdrawal of the transfer catheter 40 without excessive force.

The system accommodated in this way in the catheter is introduced into the uterine cavity until the receptacle 10 lies beyond the neck of the uterus.

The deformable element 21 is adapted, after withdrawal of the catheter, as illustrated in FIG. 3, to assume a retaining position in which it cooperates with the wall of the neck of the uterus.

As clearly illustrated diagrammatically in FIG. 3, the deformable element resumes a spiral shape inside the neck of the uterus and comes into contact with the wall of the neck of the uterus to be held in the latter by bearing engagement therewith.

Thanks to this spiral shape, the neck of the uterus is not blocked by the retaining device 20, contact between the deformable element 21 and the neck of the uterus occurring along a spiral curve against the walls of the neck of the uterus.

Thanks to this retaining device, the receptacle containing the gametes may be held in position in the uterus for the duration chosen by the obstetrician.

Moreover, as soon as the recoverable intra-uterine system is retained in the neck of the uterus, there is no risk of lesion of the endometrial wall in the uterine cavity, which could impede subsequent re-implantation and development of the embryo.

This feature is particularly important when, at the moment of implanting the embryo, it is placed in contact with the endometrial layer of the uterus to enable nidation and development of the embryo in the uterus.

The present invention is not limited to the embodiment described above, of course.

Thus the elastically deformable element 21 could be replaced by an elongate shape memory element adapted to be deformed by the effect of the heat generated in the human body, for example, or thanks to the presence of moisture.

This deformation could thus be obtained inside the neck of the uterus.

Moreover, the deformable element could be produced from an elongate steel rod disposed inside the protective sleeve instead of the coil spring.

1-9. (canceled)

10. A recoverable intra-uterine system comprising:
a receptacle adapted to contain one or more elements comprising:
an embryo, male and/or female gametes, a fertilized oocyte, a non-fertilized ovum, or a combination thereof, and
a retaining device comprising a deformable element adapted to assume at least two positions comprising:
(i) an introduction position wherein said deformable element is substantially rectilinear and is adapted to be accommodated in a catheter; and
(ii) a retaining position wherein said deformable element is adapted to bear against the wall of the neck of the uterus.

11. The recoverable intra-uterine system of claim 10, wherein in said introduction position, the receptacle is elongated along an axis and the deformable element is substantially aligned with the axis of the receptacle.

12. The recoverable intra-uterine system claim 10, wherein said deformable element comprises a spiral deformable elongate element.

12. The recoverable intra-uterine system claim 11, wherein said deformable element comprises a spiral deformable elongate element.

13. The recoverable intra-uterine system claim 10, wherein said deformable element of the retaining device is deformable between the introduction position and the retaining position by an elastic return force.

14. The recoverable intra-uterine system claim 11, wherein said deformable element of the retaining device is deformable between the introduction position and the retaining position by an elastic return force.

15. The recoverable intra-uterine system claim 12, wherein said deformable element of the retaining device is deformable between the introduction position and the retaining position by an elastic return force.

16. The recoverable intra-uterine system of claim 13, wherein the deformable element comprises a steel element comprising a rod covered with a protective sleeve.

17. The recoverable intra-uterine system of claim 16, wherein said protective sleeve comprises a silicone tube.

18. The recoverable intra-uterine system of claim 10, wherein the retaining device is located at a proximal end of the receptacle.

19. The recoverable intra-uterine system of claim 10, further comprising a thread for withdrawing said system, fastened to the retaining device.

20. A device comprising a transfer catheter and the intra-uterine system of claim 10, wherein the recoverable intra-uterine system is located inside said catheter.
